(19) United States
(12) Patent Application Publication
(10) Pub. No.: US 2005/0054102 A1
(43) Pub. Date: Mar. 10, 2005

(54) Method for differentiating stem cells into insulin-producing cells

(76) Inventors: Anna Wobus, Gatersleben (DE); Luc St-Onge, Gottingen (DE); Przemyslaw Blyszczuk, Gatersleben (DE); Ursula Hoffmann, Juhnde (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/475,021

(22) PCT Filed: Apr. 19, 2002
(86) PCT No.: PCT/EP02/04362

Related U.S. Application Data
(60) Provisional application No. 60/284,531, filed on Apr. 19, 2001.

(51) Int. Cl. C12N 5/08; C12N 15/85

(52) U.S. Cl. 435/455; 435/366

(57) ABSTRACT

The present invention relates a novel method for differentiating stem cells into insulin-producing cells by culturing such cells in specially defined media and optimally, activating one or more genes involved in beta-cell differentiation. The present invention further relates to applications in the medical (particularly diabetes) field that directly arise from the method of the invention. Additionally, the present invention relates to applications for identifying and characterising compounds with therapeutic medical effects or toxicological effects that directly arise from the method of the invention.

### FIELD OF THE INVENTION

The present invention relates to methods for differentiating stem cells into insulin-producing cells by culturing such cells in specially defined mediums and optimally, activating one or more genes involved in beta-cell differentiation. The present invention provides means for treatment of pancreatic diseases, metabolic syndrome and metabolic disorders with impaired glucose levels, for instance, but not limited to, diabetes mellitus, hyperglycaemia and impaired glucose tolerance, by transplanting said insulin-producing cells into diabetic animals and humans. The methods can further be used to generate cells for the identification and characterisation of compounds which stimulate beta-cell differentiation, insulin secretion or glucose responsiveness. Differentiated insulin-producing cells can also be used to study the toxic and other effects of exogenous compounds on beta-cell function.

### BACKGROUND OF THE INVENTION

Diabetes, hyperglycaemia and impaired glucose tolerance are endocrine disorders characterised by inadequate production or use of insulin, which affects the metabolism of carbohydrates, proteins, and lipids resulting in abnormal levels of glucose in the blood. Diabetes is a heterogeneous disease that can be classified into two major group: Type 1 diabetes (also known as Insulin-dependent diabetes, IDDM, type I, juvenile diabetes) and Type 2 diabetes (Noninsulin-dependent diabetes, NIDDM, type II, maturity-onset diabetes).

The functional unit of the endocrine pancreas is the islet of Langerhans which are scattered throughout the exocrine portion of the pancreas and are composed of four cell types: alpha-, beta-, delta-, and PP-cells. Beta-cells produce insulin, represent the majority of the endocrine cells and form the core of the islets while alpha-cells secrete glucagon and are located in the periphery. Delta-cells and PP-cells are less numerous and secrete somatostatin and a pancreatic polypeptide respectively. Insulin and glucagon are key regulators of blood glucose levels. Insulin lowers blood glucose levels by increasing its cellular uptake and conversion into glycogen. Glucagon elevates blood glucose levels by intervening in the breakdown of liver glycogen. Type 1 diabetes is characterised by an autoimmune destruction of insulin-producing beta-cells. Type 2 diabetes is characterised by insulin resistance and impaired glucose tolerance where insulin is not efficiently used or is produced in insufficient amounts by the beta-cells. Therefore, type 2 patients often require additional insulin to regulate blood glucose levels. Consequently, there is little therapeutic difference in the administration of insulin between type 1 and type 2 diabetic patients (see Fajans in Diabetes Milletus fifth editions; Porte and Sherwin, ed; Appleton & Lange pub. 1997, 1423 pp). Individuals afflicted with diabetes must inject themselves up to six times a day with insulin.

Despite insulin injections, diabetic patients develop complications and their susceptibility to strokes, blindness, amputations, kidney and cardiovascular diseases is greatly increased while their life expectancy is shortened (Nathan (1993) N. Engl. J. Med. 328:1676-1685; Group, T. D. C. a. C. T. R. (1993) N. Engl. J. Med. 329:977-986). Replacement of absent insulin-producing cells by transplantation of islets of Langerhans or insulin-producing cells is one promising therapeutic option (Luzi et al. (1996) J. Clin. Invest. 97:2611-2618; Bretzel et al. (1996) Ther. Umsch. 53:889-901) However, the availability of human donor tissue for transplantation is severely limited. An alternative option would be the use of animal tissues from pigs but serious technical problems such as long term immunosuppression and the risk of transferring a porcine pathogen such as porcine endogenous retrovirus into the human population must be solved (Butler et al. (1998) Nature 391:320-324; Bach et al. (1998) Nature Med. 4:141-144; Shapiro et al. (2000) N. Engl. J. Med. 343:230-238). One solution to this problem would be to generate a human “surrogate cell” capable of assuming the functions of the missing or malfunctioning beta-cell. Therefore, there exists a need for producing an unlimited amount of surrogate insulin-producing cells for transplantation into diabetic patients. The present invention satisfies this need by providing an easy method for inducing the differentiation of stem cells into functional insulin-producing cells.

Stem cells are undifferentiated or immature cells that can give rise to various specialised cell types. Once differentiated or induced to differentiate, stem cells can be used to repair damaged and malfunctioning organs. Stem cells can be of embryonic or adult origin. Adult or somatic stem cells have been identified in numerous different tissues such as muscle, bone marrow, liver, and brain (Vescovi and Snyder (1999) Brain Pathol., 9:569-598; Seale and Rudnicki (2000) Dev. Biol., 218:115-124). In the pancreas, several indications suggest that stem cells are also present within the adult tissue (Gu and Sarvetnick (1993) Development, 118:33-46; Bouwens (1998) Microsc Res Tech, 43:332-336; Bonner-Weir (2000) J. Mol. Endocr., 24:297-302). However, this population is poorly defined and represents a very small percentage of cells in the pancreas.

Embryonic stem cells can be isolated from the inner cell mass of pre-implantation embryos (ES cells) or from the primordial germ cells found in the genital ridges of post-implanted embryos (EG cells). When grown in special culture conditions such as spinner culture or hanging drops, both ES and EG cells aggregate to form embryoid bodies (EB). EBs are composed of various cell types similar to those present during embryogenesis. When cultured in appropriate media, EB can be used to generate in vitro differentiated phenotypes, such as extraembryonic endoderm, hematopoietic cells, neurons, cardiomyocytes, skeletal muscle cells, and vascular cells. No method has been described so far that allows EB to efficiently differentiate into insulin-producing cells.

Soria and colleagues describe a method for selecting insulin-secreting cell clones from ES cells using a cell-trapping system, wherein cells are transfected with a plasmid allowing the expression of neomycin resistance gene under the control of the regulatory region of the human insulin gene. Cells from an insulin-secreting cell clone were implanted in the spleen of diabetic mice. The implanted cells can normalise blood glucose levels and restore body weight in the treated animals (Soria et al. (2000) Diabetes 49:157-162). A disadvantage of this selection method is, however, its low efficiency.

Lumelsky and colleagues (Lumelsky et al. (May 2001), Science 292: 1389-1394) have generated insulin-expressing cells from mouse ES cells. ES cells are expanded on a gelatine-coated tissue culture surface without feeder cells and in the presence of LIF. Then, embryoid bodies are generated in suspension in ES cell medium in the absence of LIF. In a further stage nestin-positive cells are selected in a serum-free medium (ITSFn) on tissue culture surface. Resulting pancreatic endocrine progenitor cells are expanded and the differentiation and morphogenesis of insulin-secreting islet clusters is induced. However, the insulin-secreting islet clusters did not restore normal blood glucose levels when transplanted into diabetic mice.

Assady et al. (August 2001), Diabetes, 50:1-7) describe a spontaneous in vitro differentiation of pluripotent human embryonic stem cells into cells having the characteristics of insulin-producing cells. Secretion of insulin into the medium was observed in a differentiation-dependent manner and was associated with the appearance of other β-cell markers. However, the efficiency of differentiation was low with only 1-3% of differentiated cells positive for insulin.

The present invention is aimed at inducing the differentiation of ES cells by activation of specific genes into insulin-producing cells and is therefore different from the methods of the prior art designed to select such cells.

In recent years, several genes have been shown to be essential for the generation of pancreatic endocrine cells during embryogenesis (Edlund (1998) Diabetes, 47:1817-1823; St-Onge et al. (1999) Curr. Opin. Genet. Dev., 9:295-300). Pancreas development involves a series of inductive signals emanating from the surrounding mesodermic tissues and transcription factors expressed in the pancreatic epithelium. The homeobox containing transcription factor Pdx1 (also referred to Idx1, STF1, IPF1) is expressed in all cells of the pancreatic buds during development and will become restricted to the beta-cells in adult animals. Pdx1 mutant mice do not develop any exocrine nor endocrine tissue and do not have any pancreas (Jonsson et al. (1994) Nature, 371:606-609; Ahlgren et al. (1996) Development, 122:1409-1416; Offield et al. (1996) Development, 122:983-995). The basic helix-loop-helix transcription factor neurogenin3 (ngn3) is required for the specification of the early endocrine precursor in the pancreatic epithelium and is downregulated once endocrine differentiation begins (Apelqvist et al. (1999) Nature, 400:877-881; Jensen et al. (2000) Diabetes, 49:163-176; Gradwohl et al. (2000) Proc. Natl. Acad. Sci. U.S.A., 97:1607-1611). Two members of the Pax gene family, Pax4 and Pax6, are essential for proper differentiation of endocrine cells in the pancreas (Sosa-Pineda et al. (1997) Nature, 386:399-402; St-Onge et al. (1997) Nature, 397:406-409; Sanders et al. (1997) Genes Dev., 11:1662-1673). Both Pax genes are expressed early in development in a subset of endocrine precursor cells of the pancreatic epithelium, before differentiation of the mature hormone-producing cells. Mice lacking Pax4 fail to develop any beta-cells and are diabetic while the alpha-cell population is absent in Pax6 mutant mice. Nkx2.2, Nkx6.1, Nkx6.2, Isl1, and NeuroD are also among essential transcription factors required for the proper differentiation and function of beta-cells.

Several animal models for beta-cell regeneration suggest that the mechanisms involved in beta-cell differentiation in adult organism are similar to the mechanisms involved in beta-cell differentiation during embryogenesis. Gu and Savernick have established a model system for studying pancreatic islet and beta-cell regeneration in transgenic mice bearing the interferon-gamma (IFN-gamma) gene expressed in pancreatic islets. In this model, new islet cells (i.e. beta-, alpha-, delta- and PP-cells) are formed continuously from pancreatic duct cells (Gu and Savernick (1993) Development, 118:33-46). They show that duct cell proliferation and the duct-associated islet formation in IFN-gamma transgenic mice is recapitulating islet formation during development and requires the expression of Pax4, Pax6 and Pdx1 genes. Although a link exists between the genes involved in islet regeneration in adult animals and beta-cell differentiation during embryogenesis, it has not been shown in the prior art that activation of such genes in stem cells can induce the differentiation into insulin-producing cells.

### SUMMARY OF THE INVENTION

The present invention relates a novel method for differentiating stem cells into insulin-producing cells by culturing such cells in specially defined media and optimally, activating one or more genes involved in beta-cell differentiation. The present invention further relates to applications in the medical and diabetes field that directly arise from the method of the invention. Additionally, the present invention relates to applications for identifying and characterising compounds with therapeutic medical effects or toxicological effects that directly arise from the method of the invention.

### DETAILED DESCRIPTION OF THE INVENTION

Before the present methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

A technical problem underlying the present invention is to provide a method for generating insulin-producing cells for transplantation in patients afflicted with pancreatic diseases, such as for example but not limited to, hyperglycaemia, impaired glucose tolerance, gestational diabetes, and diabetes mellitus. The solution to said technical problem is achieved by the embodiments characterised in the claims.

Thus, the present invention relates to methods for differentiating stem cells into insulin-producing cells comprising
(a) Activating one or more pancreatic genes in a stem cell
(b) Aggregating said cells to form embryoid bodies
(c) Cultivating embryoid bodies in specific differentiation media enhancing beta-cell differentiation
(d) Identification and selection of insulin-producing cells and of pancreatic cells.

In connection with the present invention, the term “stem cells” denotes an undifferentiated or immature embryonic, adult or somatic cells that can give rise to various specialised cell types. The term stem cells can includes embryonic stem cells (ES) and primordial germ cells (EG) cells of human or animal origin. Isolation and culture of such cells is well known to those skilled in the art (Thomson et al. (1998) Science 282:1145-1147; Shamblott et al. (1998) Proc. Natl. Acad. Sci. USA 95:13726-13731; U.S. Pat. No. 6,090,622; U.S. Pat. No. 5,914,268; WO 0027995; Notarianni et al. (1990) J. Reprod. Fert. 41:51-56; Vassilieva et al. (2000) Exp. Cell. Res. 258:361-373). The term “stem cells” can include neural progenitor cells from embryonic, fetal or adult neural tissues. Isolation and culture of such cells is well known to those skilled in the art (Rao (Ed.), Stem Cells and CNS Development, Humana Press Inc., New Jersey (2001); Fedoroff and Richardson (Eds.), Protocols for Neural Cell Culture, Humana Press Inc., 3rd edition, New Jersey, (2001)).

The term “insulin-producing cell” means a cell capable of expressing, producing, and secreting insulin.

The term “cultivation medium” means a suitable medium capable of supporting growth and differentiation of stem cells, preferably ES and EG cells. Examples of suitable culture media in practising the present invention are prepared with a base of Dulbecco's modified Eagle's medium (DMEM, Life Technologies) supplemented with 15% heat-inactivated foetal calf serum (FCS, Gibco), and additives, such as 2 mM L-glutamine (Gibco), 5×10⁻⁶M β-mercaptoethanol (Serva) and 1:100 non-essential amino acids (Gibco). Another example is a culture medium comprising Iscove's modified Dulbecco's medium (IMDM, Gibco) supplemented with 20% FCS, 2 mM L-glutamine (Gibco), 1:100 non-essential amino acids (Gibco) and 450 μM α-monothioglycerol (Sigma). For routine cultures, ES cells are grown on a feeder layer of embryonic fibroblasts inactivated by treatment with 100 μg/ml mitomycin C for 3 hours.

The term “differentiation medium” means a suitable medium for inducing the differentiation of stem cells into insulin-producing cells. Examples of suitable culture media in practising the present invention are prepared with a base of Iscove's modified Dulbecco's medium (IMDM, Gibco) supplemented with 20% fetal calf serum (FCS), 2 mM L-glutamine, 1:100 non-essential amino acids and 450 μM α-monothioglycerol (Sigma). In addition, such medium can contain between 1 ng/ml and 100 μg/ml, preferably 10 ng/ml Epithelial Growth Factor (EGF); between 1 ng/ml and 100 μg/ml, preferably 2 ng/ml basic Fibroblast Growth Factor (bFGF); between 1 nM and 1 mM, preferably 20 nM progesterone; between 10 ng/ml and 100 μg/ml, preferably 100 ng/ml Growth hormone; between 1 nM and 100 μM, preferably 5 nM follistatin (R&D); or between 1 and 100 nM, preferably 2 nM activin (R&D). Another example of suitable culture media in practising the present invention is prepared with a base of Dulbecco's modified Eagle's medium: Nutrient Mixture F-12 (DMEM/F12, Life Technologies) supplemented with between 100 ng/ml and 100 μg/ml, preferably 5 μg/ml insulin; between 1 nM and 100 nM, preferably 30 nM sodium selenite; between 100 ng/ml and 500 μg/ml, preferably 50 μg/ml transferrin; between 100 ng/ml and 100 μg/ml, preferably 5 μg/ml fibronectin. Yet another example of suitable culture media in practising the present invention is prepared with a base of Dulbecco's modified Eagle's medium: Nutrient Mixture F-12 (DMEM/F12, Life Technologies) supplemented with between 100 ng/ml and 100 μg/ml, preferably 25 μg/ml insulin; between 1 nM and 100 nM, preferably 30 nM sodium selenite; between 100 ng/ml and 500 μg/ml, preferably 50 μg/ml transferrin; between 100 ng/ml and 100 μg/ml, preferably 5 μg/ml fibronectin; between 500 ng/ml and 100 μg/ml, preferably 1 μg laminin; between 10 μM and 500 μM, preferably 100 μM putrescine; between 1 nM and 1 μM preferably 20 nM progesterone; between 100 μM and 100 mM, preferably 10 mM nicotinamide.

In addition, extracellular matrix (ECM) proteins, such as laminin (between 0.5 and 100 μg/ml, preferably 1 μg/ml, SIGMA), or collagens, or complex mixtures of growth factors and ECM proteins of basal lamina (Matrigel R, Collaborative Research/Becton Dickinson, 1:3 dilution=stock solution, final concentration in cultures=1:10) are included to enhance the number of pancreatic cells as well as their differentiation status.

The term “terminal differentiation medium” means a suitable medium for terminal differentiation of insulin-producing cells. Examples of suitable culture media in practising the present invention are prepared with a base of Iscove's modified Dulbecco's medium (IMDM, Gibco) supplemented with 20% FCS, 2 mM L-glutamine, 1:100 non-essential amino acids and 450 μM α-monothioglycerol (Sigma). In addition, such medium can contain between 1 nM and 100 μM, preferably 2 nM Activin A; between 1 nM and 100 μM, preferably 1 nM betacellulin; between 1 ng/ml and 100 μg/ml, preferably 10 ng/ml Human Growth Factor (HGF); between 1 nM and 100 μM, preferably 10 nM Niacinamid and between 1 ng/ml and 100 μg/ml, preferably 2 ng/ml Transforming Growth Factor 2beta (TGF 2beta).

The term “pancreatic gene” means a gene or its protein product that is involved and required for pancreas development, more preferably beta-cell differentiation. Examples of such genes are Pdx1 (GenBank accession number AH005712), Pax4 (GenBank accession numbers XM004974, NM006193), Pax6 (GenBank accession number M93650), ngn3 (GenBank accession numbers XM005744, NM020999, AJ133776), Nkx6.1 (GenBank accession number AH007313), Nkx6.2, Nkx2.2 (GenBank accession number AF019415), HB9 (GenBank accession numbers XM049383, AF107457), BETA2/NeuroD (GenBank accession numbers NM002500, XM002573), Isl1 (GenBank accession number NM002202), HNF1-alpha, HNF1-beta (GenBank accession number X71346), and HNF3 (GenBank accession numbers AF176112, AF176111) of human or animal origin. Preferred genes are Pdx1, Pax4, Pax6, and ngn3. Especially preferred genes are Pdx1, Pax4, and Pax6. Each gene can be used individually or in combination.

The term “activating one or more pancreatic gene” means delivering and introducing said pancreatic genes or proteins into stem cells.

In a preferred embodiment, the cDNA of one or more pancreatic genes is placed under the control of a regulatory region allowing the initiation of transcription and introduced into a cell by transfection methods such as electroporation, lipofection, calcium phosphate mediated, DEAE dextrans, and the like. Such methods and system are well described in the art and do not require any undue experimentation; see, for example, Joyner, “Gene Targeting: A Practical Approach”, Oxford University Press, New York, 1993; Mansouri “Gene Targeting by Homologous Recombination in Embryonic Stem Cell”, Cell Biology: A Laboratory Handbook, second ed., Academic Press, 1998. Gene expression of pancreatic gene can be assured by constitutive promoters such as the Cytomegalovirus promoter/enhancer region or inducible promoters such as the tetracycline inducible system. Expression vectors can also contain a selection agent such as the neomycin, hygromycin or puromycin resistance genes. Making such gene expression vectors are well known in the art; see Sambrook et al., “Molecular Cloning, A laboratory Manual” third ed., CSH Press, Cold Spring Harbor, 2000; Gossen and Bujard, (1992) Proc. Natl. Acad. Sci. USA 89:5547-5551). DNA transfer can also be achieved using a viral delivery system such as retrovirus, adenovirus, adeno-associated virus and lentivirus vectors.

In a further preferred embodiment, protein products of pancreatic genes can be delivered directly to stem cells. For example, protein delivery can be achieved by polycationic liposomes (Sells et al. (1995) Biotechniques 19:72-76), Tat-mediated protein transduction (Fawell et al. (1993) Proc. Natl. Acad. Sci. USA 91:664-668) and by fusing a protein to the cell permeable motif derived from the PreS2-domain of the hepatitis-B virus (Oess and Hildt (2000) Gene Ther. 7:750-758). Preparation, production and purification of such proteins from bacteria, yeast or eukaryotic cells are well known by persons skilled in the art.

An additional embodiment of the present invention relates to a method for aggregating stem cells, preferably ES and EG cells, to form embryoid bodies. Embryoid bodies can be generated by a hanging drop method. For example, between 400-800 ES cells, preferably 600, are cultured in drops of 20 μl of Iscove modified Dulbecco's medium (IMDM, Gibco) supplemented with 20% FCS, L-glutamine, non-essential amino acids and α-monothioglycerol placed on the lids of petri dishes filled with phosphate-buffered saline (PBS). Embryoid bodies are cultured in hanging drops for 2 days at 37° C. with 5% CO₂ and then transferred to bacteriological petri dishes (Greiner, Germany) and incubated a further 3 days in suspension culture. After 5 days, embryoid bodies are plated onto gelatin-coated 24-well plates, petri dishes or other suitable culture container and cultured for an additional 15 to 35 days at 37° C. with 5% CO₂. Embryoid bodies can also be produced in spinner cultures. For example, adherent stem cells are enzymatically dissociated using 0.2% trypsin and 0.05% EDTA in PBS (Life Technologies) and seeded at a density of 10⁷ cell/ml in 250 ml siliconised spinner flasks (Life Technologies) containing 100 culture medium. After 24 hours, 150 ml culture medium is added to a final volume of 250. Spinner flasks are stirred at 20 rpm using a stirrer system (Integra Biosciences). Such methods are well known in the art and can be scaled up for industrial production without undue experimentation.

In a further embodiment of the invention, embryoid bodies are plated unto petri dishes containing differentiation medium and allowed to differentiate into insulin-producing cells for periods of 15 to 50 days, preferably 20 to 25 days (depending on the cell lines used; R1 wild type cells need longer differentiation for generating insulin or glucagon-positive cells than Pdx-1⁺ or Pax4⁺ cells). In the method of the invention a high proportion of insulin-producing cells is obtained. After a differentiation time of 15 days, the proportion of insulin-producing cells is preferably at least 20%, more preferably at least 40% and most preferably at least 50%.

The proportion of insulin-producing cells may further be increased by a selection of nestin-positive cells. This selection preferably comprises the transfer of embryoid bodies, e.g. obtained by the hanging drop method, to a suspension culture and subsequent plating and/or replating on a suitable medium, e.g. a poly-L-ornithine/laminin coated plate. The nestin selection procedure may lead to a further increase in the proportion of insulin-producing cells, e.g. a proportion of 70% or more.

In a further embodiment of the invention, differentiated insulin-producing cells can be isolated and purified using a method for selecting insulin secreting cell clones from ES cells by transfecting cells with a plasmid allowing the expression of neomycin, hygromycin or puromycin resistance gene under the control of the regulatory region of the human insulin gene. Cells can also be sorted using Fluorescent Activated Cell Sorting (FACS) after Hoechst 33342 dye staining (Goodell et al. (1996) J. Exp. Med. 183:1797-1806). Further modifications of the above-mentioned embodiment of the invention can easily be devised by the person skilled in the art, without undue experimentation from this disclosure.

An additional embodiment of the present invention relates to a method for treating diabetes wherein between 3000 and 100 000 equivalent differentiated insulin-producing cells per kilogram body weight would be introduced into a diabetic patient intraportally via a percutaneous transhepatic approach using local anaesthesia. Such surgical techniques are well known in the art and can be applied without any undue experimentation, see Pyzdrowski et al, “Preserved insulin secretion and insulin independence in recipients of islet autografts” New England J. Medicine 327:220-226, 1992; Hering et al., “New protocol toward prevention of early human islet allograft failure” Transplantation Proc. 26:570-571, 1993; Shapiro et al., “Islet transplantation in seven patients with type 1 diabetes mellitus using a glucocorticoid-free immunosuppressive regimen”, New England J. Medicine 343:230-238, 2000. Furthermore, encapsulation technology could also be used for the transplantation of differentiated insulin-producing cells as described by Lanza et al., “Encapsulated cell technology”, Nature Biotech 14:1107-1111, 1996.

Further, the invention relates to a cell composition comprising differentiated stem cells exhibiting insulin production, e.g. an insulin-producing cell line obtainable by the method as described above. The insulin-producing cells may exhibit a stable or a transient expression of at least one gene involved in β-cell differentiation, particularly a gene as described above. The cells are preferably human cells which are derived from human stem cells. For therapeutic applications the generation of autologous human cells from adult stem cells of a patient is especially preferred.

The insulin-producing cells of the invention exhibit characteristics which closely resemble naturally occurring β-cells. Particularly, the ratio of insulin-producing cells versus glucagon-producing cells is high. After 15 days of differentiation, this ratio is preferably at least 2:1 and more preferably at least 5:1. Further, the cells of the invention are capable of a quick response to glucose. After addition of 27.7 mM glucose, the insulin production is enhanced by a factor of at least 2, preferably by a factor of at least 3 in the cells of the invention. Further, the cells of the invention are capable of normalizing blood glucose levels after transplantation into mice.

The cell composition of the invention is preferably a pharmaceutical composition comprising the cells together with pharmacologically acceptable carriers, diluents and/or adjuvants. The pharmaceutical composition is preferably used for the treatment of diabetes. The administration is preferably by transplantation as described above.

In a further embodiment, the present invention allows the generation of cells for the identification and/or characterisation of compounds which stimulate beta-cell differentiation, insulin secretion or glucose response. This method is particularly suitable for in vivo testing for diagnostic applications and drug development or screening. The compound of interest is added to differentiated and undifferentiated insulin-producing cells which are grown in appropriate culture system, for example 96 and 384 well plates. Insulin levels in treated cells can be quantified by Enzyme Linked Immunoabsorbent Assay (ELISA) or Radio Immuno Assay (RIA). Using this method, a large number of compounds can be screened and compounds that induce beta-cell differentiation and increase insulin secretion can be identified readily.

Preferred embodiments for high-throughput screening and medium throughput validation methods are described in FIG. 11. In a high-throughput screening method, the cells are transfected with a DNA construct, e.g. a viral or non-viral vector containing a reporter gene, e.g. the lacZ gene or the GFP gene, under regulatory control of a promoter of a gene involved in β-cell differentiation, e.g. a promoter of a gene as described above, preferably a Pax4 promoter. The transfected cells are divided into aliquots and each aliquot is contacted with a test substance, e.g. candidate 1, candidate 2 and candidate 3. The activity of the reporter gene corresponds to the capability of the test compound to induce β-cell differentiation.

In a further embodiment (which may be combined with the high-throughput screening as described above) a medium throughput validation is carried out. Therein, the test compound is added to stem cells being cultivated and the insulin production is determined. Following an initial high throughput assay, such as the cell based assay outlined above where e.g. a Pax4 promoter is used as marker for beta-cell regeneration, the activity of candidate molecules to induce beta-cell differentiation is tested in a validation assay comprising adding said compounds to the culture media of the embryoid bodies. Differentiation into insulin-producing cells is then evaluated, e.g. by comparison to wild type and/or Pax4 expressing ES cells to assess the effectiveness of a compound.

### BRIEF DESCRIPTION OF THE DRAWINGS

FIG. 1: Expression vectors containing the Pdx1, Pax4, Pax6, and ngn3 gene.

The Pdx1, Pax4, Pax6, and ngn3 (SEQ ID No. 1, 2, 3, 4) cDNA were inserted into the expression vector pACCMV.pLpA previously described by Becker et al. (Becker et al. (1994) Meth. Cell Biol. 43:161-189). Briefly, a Kpn I-Bam HI fragment that included the Pdx1 cDNA (SEQ ID No. 1) was introduce into the KpnI-BamHI sites of pACCMV.pLpA, placing the Pdx1 gene under the control of the Cytomegalovirus (CMVp) promoter. Likewise, a Bam HI-Hind III fragment that include the Pax4 cDNA (SED ID No. 2) was introduce into the Bam HI-Hind III sites of pACCMV.pLpA, placing the Pax4 gene under the control of the CMV promoter; a Bam HI-Hind III fragment that includes the Pax6 cDNA (SED ID No. 3) was introduced into the Bam HI-Hind III sites of pACCMV.pLpA, placing the Pax6 gene under the control of the CMV promoter; and a Bam HI-Xba I that includes the ngn3 cDNA (SEQ ID No. 4) was introduced into the Bam HI-Xba I sites of pACCMV.pLpA, placing the ngn3 gene under the control of the CMV promoter. Abbreviations: B, Bam HI; H, Hind III; K, Kpn I; X, Xba I; Ad 5, adenovirus type 5.

FIG. 2. Differentiation of ES cells into insulin-producing cells

Wild type and Pdx1 expressing embryonic stem (ES) cells were cultivated as embryoid bodies (EB; EBs) by the hanging drops method. Differentiation and terminal differentiation media are applied upon plating of EBs.

FIG. 3. Amount of hormone-producing cells in Pdx1+ differentiated ES cells

Immunofluorescence observation of insulin, glucagon, pancreatic polypeptide (PP) and somatostatin-positive cells following plating of Pdx1+ embryoid bodies cultured in normal culture medium and differentiation medium. Results illustrated over time in arbitrary units representing the average number of hormone-producing cells in define areas of the culture dishes. The number of hormone-producing cells (i.e. insulin, glucagon, PP, and somatostatin) is higher when embryoid bodies are cultured in differentiation and terminal differentiation media.

FIG. 4. Expression of pancreas specific genes after differentiation of wild type, Pdx1⁺, and Pax4⁺ ES cells into insulin-producing cells.

mRNA levels of pancreas specific genes following formation of embryoid bodies by the hanging drop method and plating in differentiation medium. Insulin and Glut2 levels are higher in Pdx1⁺ and Pax4⁺ ES cells than in wild type ES cells indicating that differentiation is more efficient when a pancreatic developmental control gene is activated.

FIG. 5. Differentiation of mouse ES cells into insulin-producing cells.

The proportion of insulin-producing cells was determined in wild type cells (R1), and Pdx1 and Pax4 expressing cells, 5, 6, 10, and 15 days after plating.

FIG. 6. Insulin-producing cells versus glucagon-producing cells.

The expression of insulin and glucagon in wild type ES cells, Pdx1 expressing cells and Pax4 expressing cells was determined 5, 10 or 15 days after plating.

FIG. 7. Glucose response of Pax4 ES cell derived insulin cells.

The insulin secretion of wild type (R1) and Pax4 ES derived insulin-producing cells was determined in the absence of glucose and 15 minutes after stimulation with 27.7 mM glucose.

FIG. 8. Regulation of blood glucose level in diabetic mice.

The blood glucose level of diabetic control mice (STZ control) and diabetic mice having received a transplant of insulin-producing cells derived from Pax4 ES cells was determined.

FIG. 9. Drug screening strategies.

A high-throughput screening and a medium throughput validation method for three test compounds are shown. An initial high throughput screen is performed in a cell assay using Pax promoters as reporter for beta-cell differentiation. Positive candidates are then validated in a medium throughput assay involving embryoid bodies. Compounds are tested at different stages of culture for their potential to induce the formation of insulin-producing cells.

FIG. 10. Differentiation methods of ES cells into insulin-producing cells using culture conditions favouring the formation of nestin-positive cells.

FIG. 11. Differentiation of nestin-positive mouse ES cells into insulin-producing cells.

### EXAMPLES

A better understanding of the present invention and of its many advantages will be had from the following examples, given by way of illustration.

### Example 1

Generation of ES Cells Expressing the Pdx1 or Pax6 Gene.

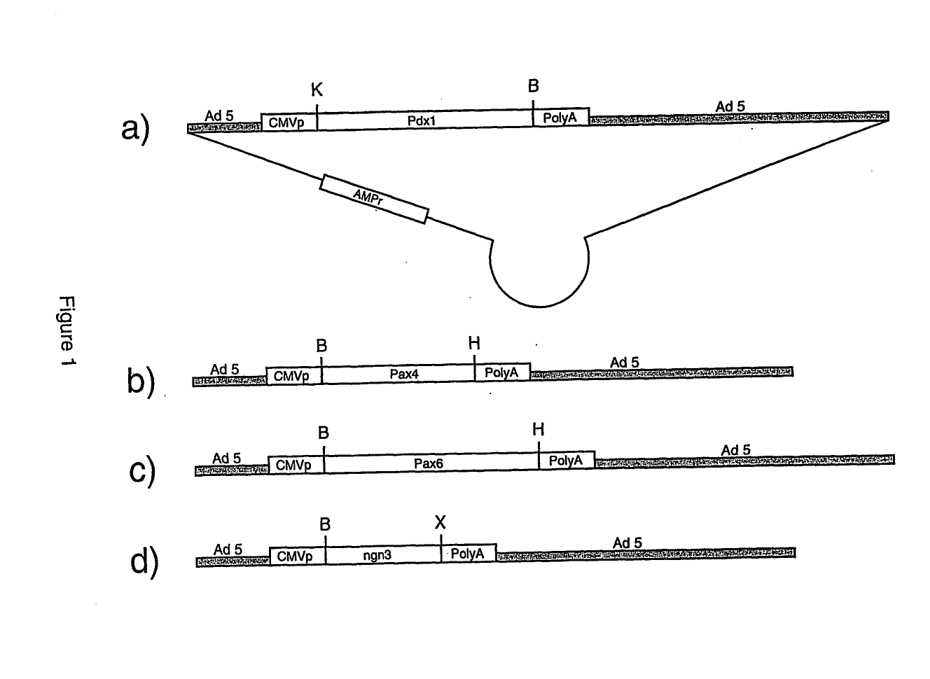

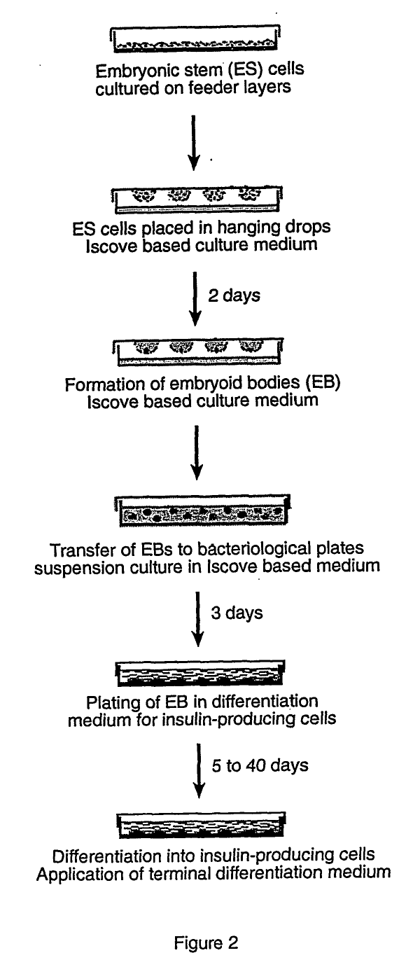

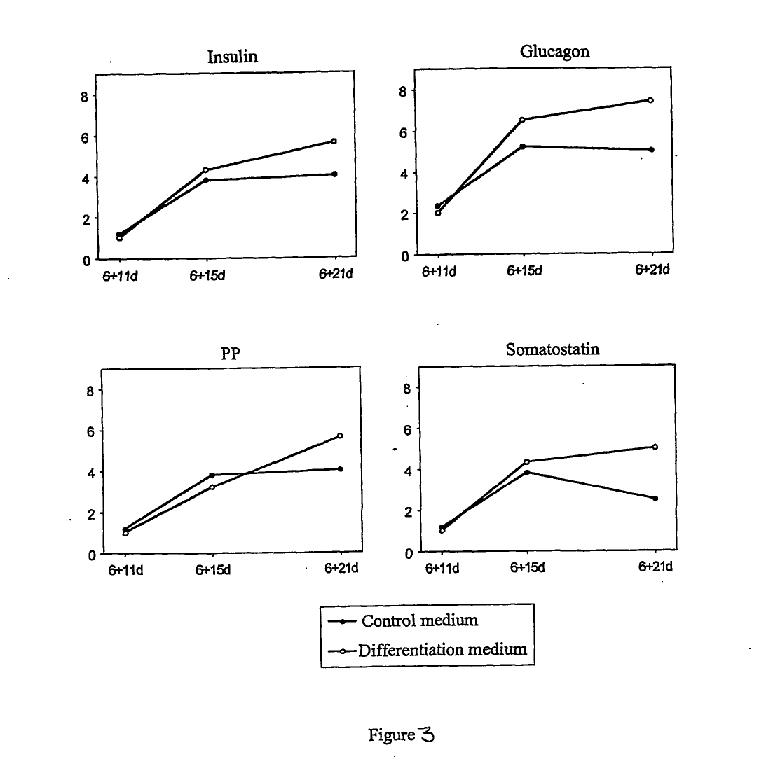

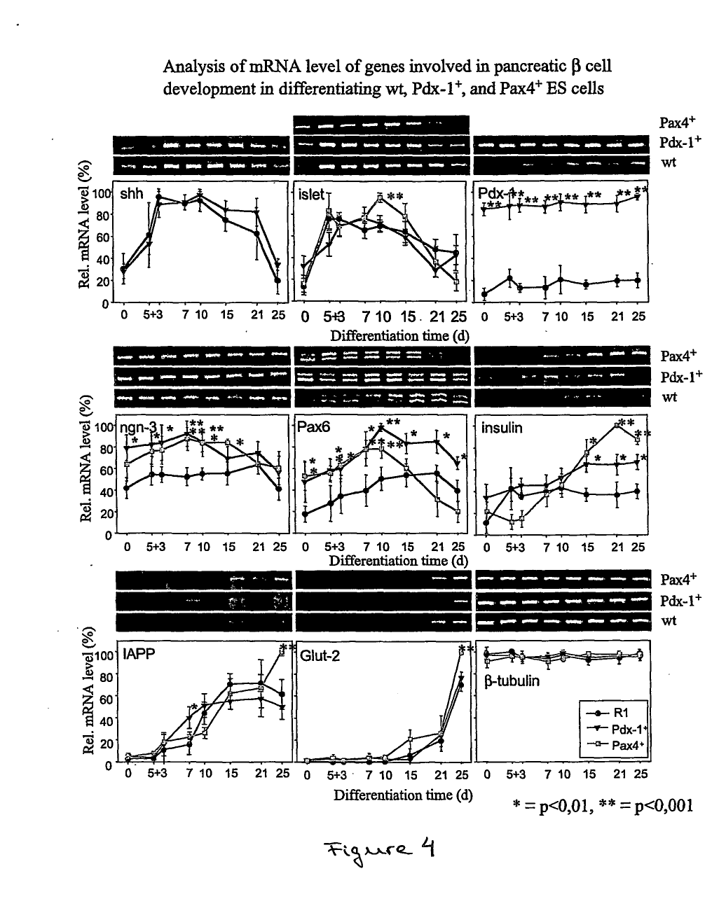

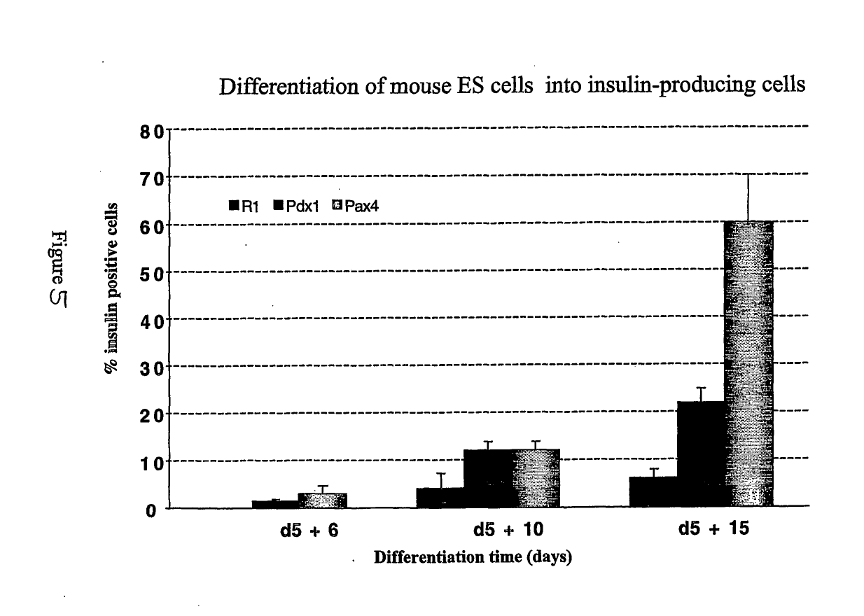

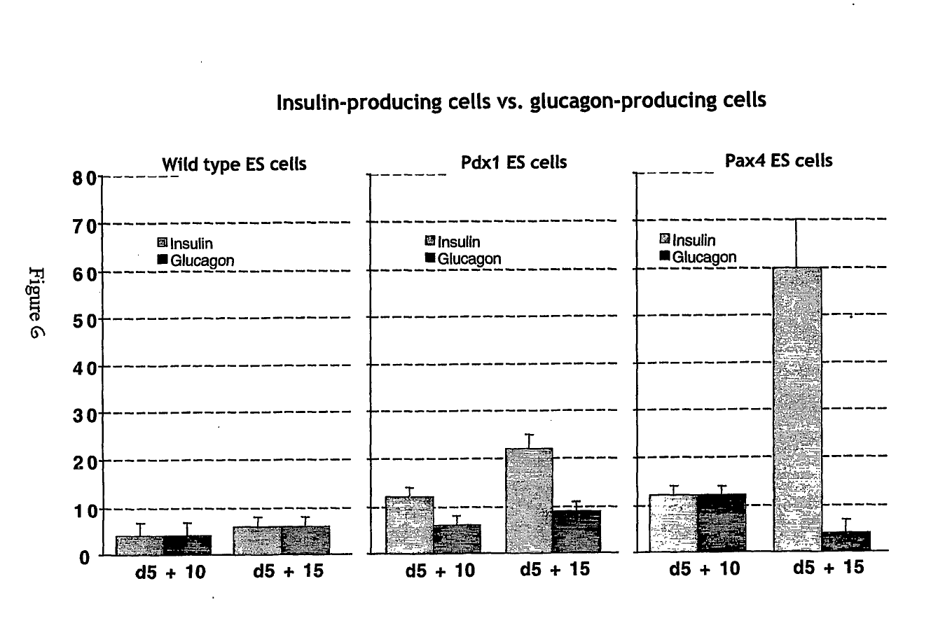

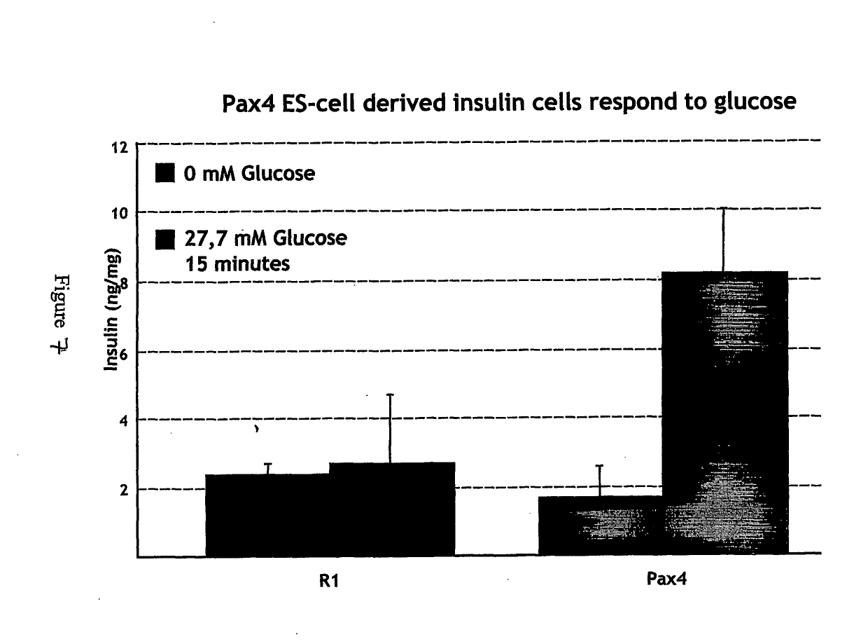

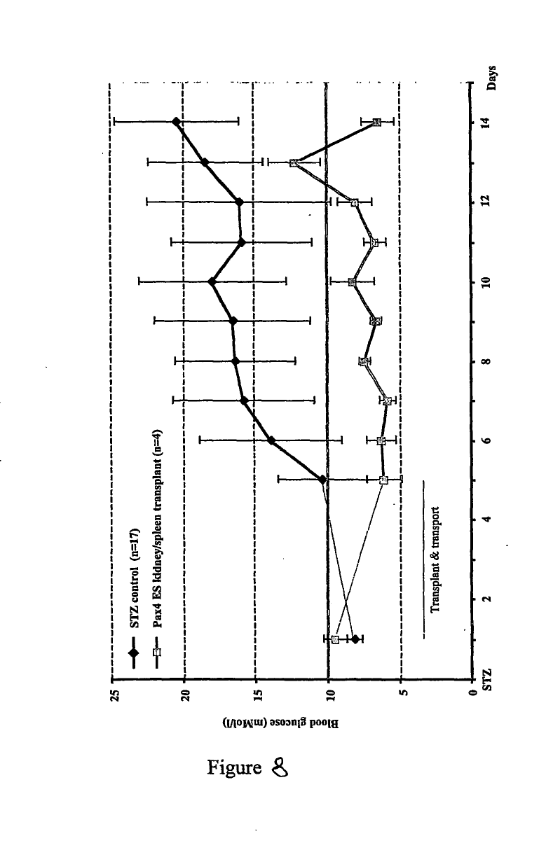

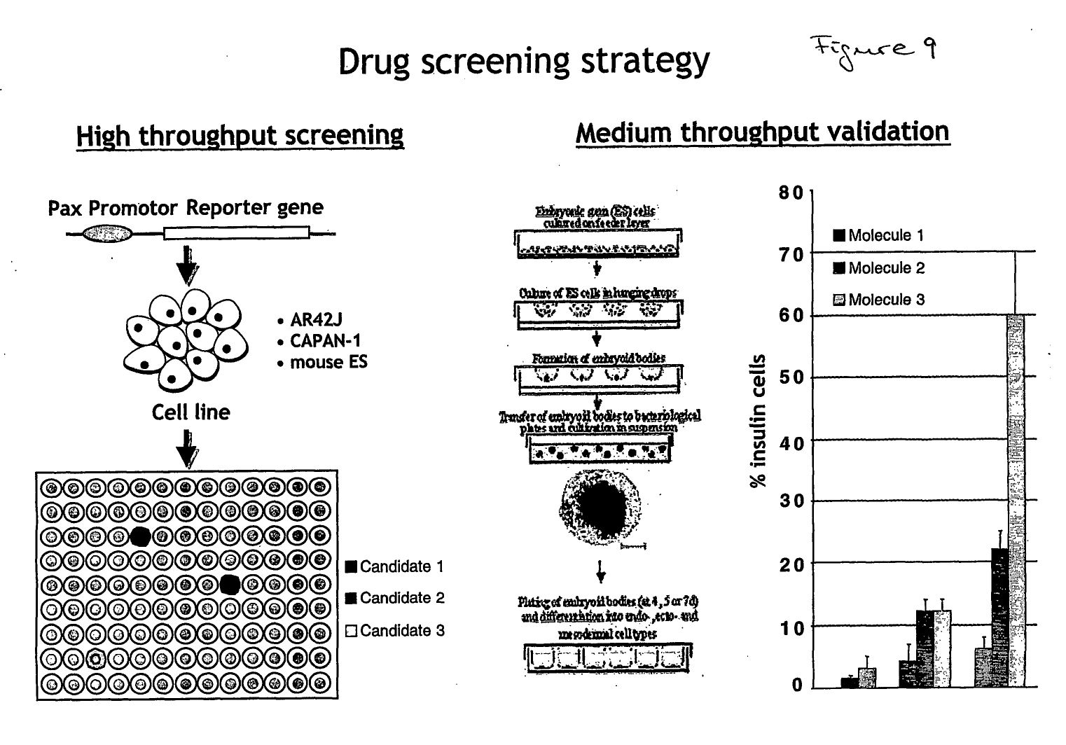

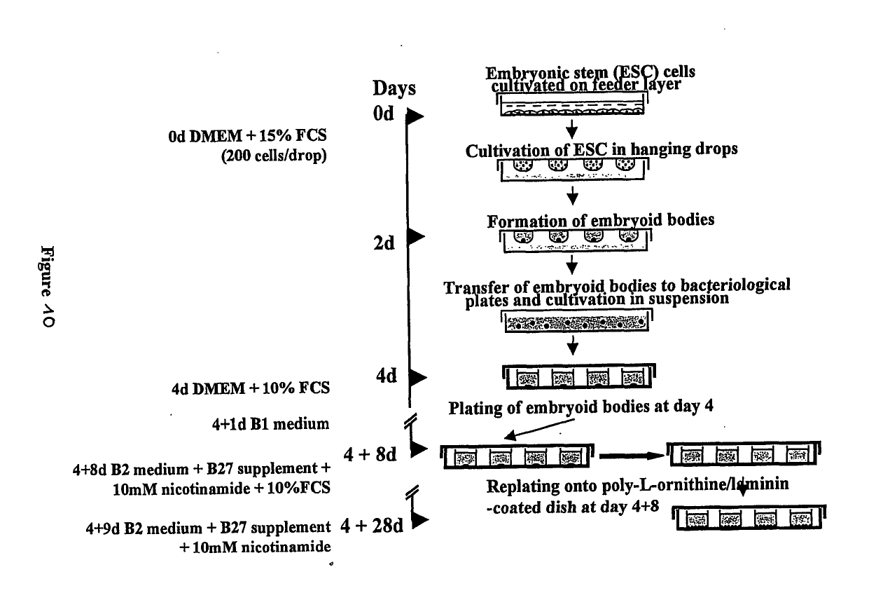

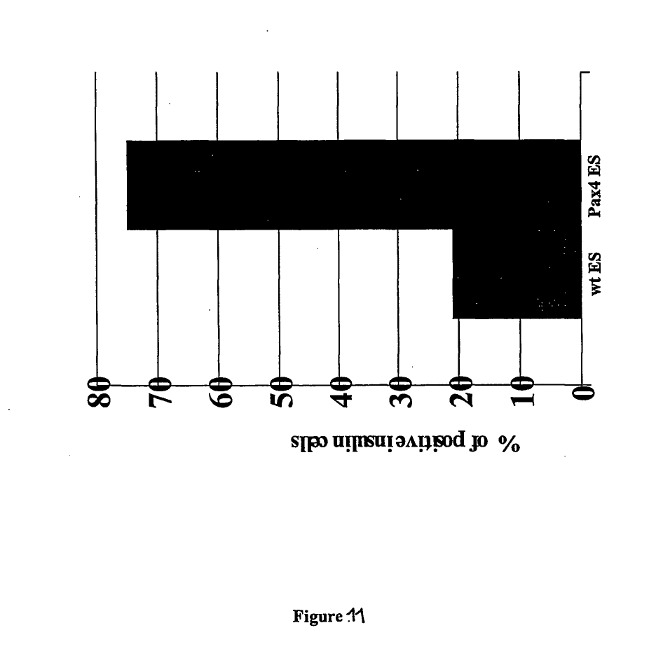

The mouse R1 ES cells (Nagy et al. (1993) Proc. Natl. Acad. Sci. USA. 90:8424-8) were electroporated with the Pax6 or the Pdx1 gene under the control of the CMV promoter (see FIG. 1) and the neomycin resistance gene under the control of the phosphoglycerate kinase I promoter (pGK-1). ES cells are cultured in Dulbecco's modified Eagle's medium (DMEM, Life Technologies) containing 4.5 g/l glucose, 10⁻⁴ M beta-Mercaptoethanol, 2 nM glutamine, 1% non essential amino acids, 1 nM Na-pyruvate, 15% FCS and 500 U/ml leukaemia inhibitory factor (LIF). Briefly, approximately 10⁷ ES cells resuspended in 0.8 ml phosphate buffered saline (PBS) containing 25 μg/ml of linearized expression vector and electroporated with one pulse of 500 μF and 250 volts at room temperature using a Gene Pulser electroporation apparatus (BioRad). Five minutes after electroporation, ES cells are plated on 8.5 cm petri dishes containing fibroblastic feeder cells previously inactivated by treatment with 100 μg/ml mitomycin C for 3 hours. One day after electroporation, culture medium is changed to medium containing 450 μg/ml G418. Resistant clones are separately isolated and cultured 14 days after applying the selection medium. Cells are always cultured at 37° C., 5% CO₂.

### Example 2

Differentiation of ES Cells into Insulin-Producing Cells.

The ES cell line R1 (wild type, wt) and ES cells constitutively expressing Pdx1 (Pdx1+) were cultivated as embryoid bodies (EB; EBs) by the hanging drops method (FIG. 2). Briefly, approximately 600 cells were placed in drops of 20 μl medium composed of Iscove modified Dulbecco's medium (IMDM, Gibco) supplemented with 20% FCS, L-glutamine, non-essential amino acids and alpha-monothioglycerol (Sigma, Steinheim, Germany; final concentration 450 μM). Drops were placed on the lids of petri dishes filled with phosphate-buffered saline (PBS). The EBs were allowed to form in hanging drops cultures for 2 days and then transferred for three days to suspension cultures in bacteriological petri dishes (Greiner, Germany). At day 5, EBs were plated separately onto gelatin-coated 24-well plates containing a differentiation medium prepared with a base of Iscove modified Dulbecco's medium (IMDM, Gibco) supplemented with 20% FCS, 2 mM L-glutamine, 1:100 non-essential amino acids, 450 μM α-monothioglycerol (Sigma), 10 ng/ml Epithelial Growth Factor (EGF, R&D Research), 2 ng/ml basic Fibroblast Growth Factor (bFGF, R&D Research), 20 nM progesterone (R&D Research), 100 ng/ml Human Growth Hormone (HGH, R&D Systems) and 5 nM follistatin (R&D Systems) and/or 2 nM human activin A (R&D Systems). Cells were cultured for 15 to 40 days in the differentiation medium. To enhance differentiation capacity, a terminal differentiation medium can be applied at stages between 5 and 20 days after EB plating.

### Example 3

Hormonal Expression in Differentiated ES Cells.

Expression of insulin, glucagon, somatostatin and pancreatic polypeptide (PP) was verified by immunofluorescence in differentiated wt and Pdx1+ ES cells. Immunofluorescence was performed according to standard protocols (see Wobus et al.: In Vitro Differentiation of Embryonic Stem Cells and Analysis of Cellular Phenotypes, In: Tymms, M. J. and Kola, I. (Eds.) Gene Knockout Protocols, vol. 158, Methods in Molecular Biology, Humana Press, Totowa, N.J., 2001). Briefly, differentiated wt or Pdx1+ ES cells are grown on cover slips and rinsed twice with PBS and fixed with methanol: acetone 7:3 at −20° C. for 10 min. The following antibodies were used: Mouse anti-insulin (Sigma-Aldrich Co.), rabbit anti-glucagon (Dako Corporation), rabbit anti-somatostatin (Dako Corporation), rabbit anti-PP (Dako Corporation) were used as primary antibody while Fluorescein (DTAF)-conjugated goat anti-mouse IgG (Jackson ImmunoResearch Laboratories) and Cy^{3TM}-conjugated goat anti rabbit IgG (Jackson ImmunoResearch Laboratories) were used as second antibody. In this study double immunostaining was performed, and the following pairs of antibodies were used: anti-insulin and anti-glucagon; anti-insulin and anti-somatostatin; anti-insulin and anti-PP. Cells were analyzed with a fluorescence microscope Optiphot-2 (Nikon) and a confocal laser scanning microscope (CLSM) LSM-410 (Carl Zeiss). Differentiated wt ES cells co-express insulin, glucagon, PP, and somatostatin indicating that the cells have not undergone maturation into single hormone-producing cells. However, differentiated Pdx1+ cells separately express either insulin or glucagon but, rarely both hormones at the same cells demonstrating that such cells achieve maturation into single hormone-producing cells. The number of hormone-producing cell is higher when Pdx1+ ES cells are cultured in a differentiation medium (see FIG. 3) illustrating that differentiation into insulin-producing cells is more efficient when a pancreatic developmental control gene is expressed in a stem cell (e.g. ES) and when such cells are cultured in a differentiation medium.

### Example 4

Expression of Pancreas Specific Genes after Differentiation of ES Cells into Insulin-Producing Cells.

Expression levels of pancreas specific genes was measured by semi-quantitative RT-PCR analysis. Differentiated wild type, Pdx-1⁺ and Pax4⁺ cells have been collected after embryoid body formation (5d) and 2, 7, 10, 15, 21 and 24 days after plating (5+2d, +7d, +10d, +15d, +21d, +24d) were suspended in lysis buffer (4 M guanidinium thiocyanate, 25 mM sodium citrate, pH 7; 0.5% sarcosyl, 0.1 M beta-mercaptoethanol). Total RNA was isolated by the single step extraction method described by Chomczynski and Sacchi (Chomczynski and Sacchi (1987) Anal. Biochem. 162: 156-159). mRNA was reverse transcribed using PolyT tail primer Oligo d(T)₁₆ (PerkinElmer) and the resulting cDNA was amplified using oligonucleotide primers complementary and identical to transcripts of the following genes: GLUT2 (SEQ ID No 9 and 10; annealing temperature 60° C. for 40 cycles, expected fragment size 556 bp), insulin (SEQ ID No 11 and 12; annealing temperature: 60° C. for 40 cycles, expected fragment size 340 bp), ngn-3 (SEQ ID No 13 and 14; annealing temperature: 60° C. for 40 cycles, expected fragment size 514 bp), Pdx-1 (SEQ ID No 15 and 16; annealing temperature: 60° C. for 45 cycles, expected fragment size 230 bp) and Isl1 (SEQ ID No 17 and 18; annealing temperature: 60° C. for 40 cycles, expected fragment size 514 bp). The house keeping gene beta-tubulin (SEQ ID No 19 and 20, annealing temperature: 60° C. for 28 cycles, expected fragment size 317 bp) was used as internal standard. Reverse transcription (RT) was performed with MuLV reverse transcriptase (Perkin Elmer). Multiplex PCRs were carried out using AmpliTaq DNA polymerase (Perkin Elmer) as described in Wobus at al., 1997. Briefly, RT reactions (20 μl) were performed with MuLV reverse transcriptase. Separate PCRs using primers of the analysed genes or primers of the house keeping gene beta-tubulin were carried out with 3 μl of the RT products. mRNA levels of genes encoding Pax4 and insulin were analysed using the Dynalbeads mRNA DIRECT micro kit (Dynal) according to the manufacturer's instructions.

One third of each PCR reaction was separated by electrophorese on 2% agarose gels containing 0.35 μg/ml of ethidium bromide. Gels were illuminated with UV light and the ethidium bromide fluorescence signals of gels were stored by the E.A.S.Y. system (Herolab) and analyzed by the TINA2.08e software (Raytest Isotopenmeβgeräte GmbH). The intensity of the ethidium bromide fluorescence signals was determined from the area under the curve for each peak and the data of target genes were plotted as percentage changes in relation to the expression of the housekeeping gene beta-tubulin.

Results show that markers for beta-cell differentiation function were expressed at higher levels in Pdx1⁺ and Pax4⁺ differentiated ES cells than in differentiated wild type ES cells demonstrating that activation of a pancreatic developmental control gene renders differentiation more efficient than for wild type ES cells (FIG. 4). Expression of GLUT2 in differentiated stem cells indicates that hormone-producing cells are capable of responding to glucose. In addition, genes involved in early endodermal/pancreatic precursor cell specification such as ngn3 and Isl1 are downregulated in Pdx-1⁺ and Pax4⁺ ES cells, consistent with in vivo data indicating that such cells have matured into single hormone-producing cells.

### Example 5

Hormonal Expression of Differentiated ES Cells Expressing Pdx1 and Pax4

In order to study the potential of pancreatic developmental control to induce beta-cell differentiation in vitro, we have generated stable mouse embryonic stem (ES) cells expressing the Pax4 or Pdx1 gene under the control of the cytomegalovirus (CMV) early promoter/enhancer region (see FIG. 1a,b). The CMV-Pax4 and CMV-Pdx1 transgenes were introduced into ES cells by electroporation, a method that is well known in the art, for example see Joyner, “Gene Targeting: A Practical Approach”, Oxford University Press, New York, 1993; Mansouri “Gene Targeting by Homologous Recombination in Embryonic Stem Cell”, Cell Biology: A Laboratory Handbook, second ed., Academic Press, 1998. Pax4, Pdx1 and wild type ES cells were then cultured in hanging drops or spinner cultures to allow the formation of embryoid bodies. Embryoid bodies were subsequently plated and cultured in a differentiation medium containing various growth factors. Under such conditions, insulin-producing cells can be detected in Pdx1 and Pax4 expressing cells six days after plating (FIG. 5). By comparison, wild type ES cells did not contain any insulin-producing cell at the same stage. Ten days after plating, 12% of Pdx1 and Pax4 expressing cells were positive for insulin while the first insulin-producing cells are observed in wild type ES cells. At day 15 of plating, up to 60% of the Pax4 ES cells are positive for insulin compared to 22% for Pdx1 ES cells and 6% for wild type ES cells. These data demonstrate that Pax4, and to some extent Pdx1, can significantly promote, and enhance ES cells differentiation into insulin-producing cells compared to wild type ES cells.

The expression of Pax4 also affects the differentiation status of the insulin-producing cell. During embryogenesis, the first hormone-producing cells to arise in the developing pancreas co-express both insulin and glucagon. These cells subsequently differentiate and mature into single hormone-producing cells. In a similar fashion, all insulin-producing cells obtained from wild type ES cells also co-express glucagon suggesting that differentiation of the cells is arrested at a premature stage (FIG. 6). Such cells most likely have little therapeutic value since insulin and glucagon have opposing effect on blood glucose levels in an organism. However in Pax4 ES cells, single insulin-producing cells are generated in substantial amounts (FIG. 6). Insulin-glucagon co-expressing cells are detected in small numbers and most likely represent an ongoing differentiation process within the cultures. This observation demonstrate that Pax4 induces, and enhances the differentiation of insulin-producing cells which are more mature than the cells observed in wild type ES cells.

### Example 6

Functional Characterisation of the Differentiated Insulin-Producing Cells.

One important property of beta-cells is glucose responsive insulin secretion. To test whether the Pax4 derived insulin-producing cells possessed this glucose responsive property, in vitro glucose responsive assay was performed on the differentiated cells. Briefly, between 10 and 14 embryoid bodies were cultured in 3 cm petri dishes containing the above mentioned differentiation medium. On the day of the assay, the differentiation medium was removed and the cells were washed 3 times with Krebs Ringer Bicarbonate Hepes Buffer (KRBH; 118 mM NaCl, 4.7 KCl, 2.5 mM CaCl₂, 1.2 mM KH₂PO₄, 1.2 mM MgSO₄, 24.6 NaHCO₃, 10 mM Hepes, 2 mg/ml BSA). Cells were then incubated in 750 μl KRBH for 45 minutes at 37° C. The KRBH was then kept for measurement of basal insulin secretion and 750 μl KRBH containing 27.7 mM glucose was added to the cells. After 15 minutes incubation at 37° C., the KRBH was removed from the cells for measurement of glucose induce insulin secretion. Insulin levels were determined by Enzyme-Linked Immunosorbent Assay (ELISA) for mouse insulin (Mercodia) and performed according to the manufacturer's recommendations. An alternative medium for proper insulin release is medium based on DMEM with glucose concentration of 1 g/l (Gibco) supplemented with non-essential amino acids (Gibco, stock solution 1:100) and additional factors mentioned above. Such medium can be applied 1 to 6 days before use of the cells.

A basal insulin secretion is observed when both wild type and Pax4 ES derived insulin-producing cells are cultured in the absence of glucose (FIG. 7). However, only the Pax4 ES derived insulin-producing cells respond to glucose stimulation. In the presence of glucose, a five fold increase in insulin secretion is seen in Pax4 ES derived insulin-producing cells. Wild type ES derived insulin-producing cells do not respond to glucose.

### Example 7

Transplantation of Pax4 ES Derived Insulin-Producing Cells in STZ Diabetic Mice.

The therapeutic potential of Pax4 ES derived insulin-producing cells to improve and cure diabetes was investigated by transplanting the cells into streptozotocin induced diabetic mice. Streptozotocin is an antibiotic which is cytotoxic to beta-cells when administered at certain dosage (see Rodrigues et al.: Streptozotocin-induced diabetes, in McNeill (ed) Experimental Models of Diabetes, CRC Press LLC, 1999). Its effect is rapid, rendering an animal severely diabetic within 48 hours.

Non-fasted Male BalbC mice were treated with 170 mg/Kg body weight STZ. Under such conditions, 17 control mice developed hyperglycaemia 6 days after STZ treatment. Mice are considered diabetic if they have a blood glucose level above 10 mMol/l for more than 3 consecutive days. One mouse did not respond to the STZ treatment. Elevated blood glucose levels varied significantly between animals and between days. This is indicative of diabetes since the animals cannot regulate their blood glucose. Cells were transplanted under the kidney capsule and into the spleen of animals. Briefly, mice were anaesthetised by intraperitoneal injection of 15 μl/g body weight avertin (2.5% tribromoethyl alcohol:tertiary amyl alcohol). The kidney and the spleen was exposed through a lumbar incision, and cells were transferred into each tissue using a blunt 30G needle.

Transplantation of cells under the kidney capsule and into the spleen were performed 24-48 hours after STZ treatment. 8 animals were transplanted with between 1×10⁶ and 5×10⁶ Pax4 ES derived insulin-producing cells. 4 out of 8 transplanted animals died due to the surgical procedure. Of the 4 animals that did survived, none developed diabetes when compared with STZ-treated control animals (FIG. 8). The presence of the insulin-producing cells was confirmed by immunohistological analysis of the transplanted tissue. These results demonstrate that the transplanted cells can normalise blood glucose in diabetic animals.

### Example 8

Differentiation of ES Cells into Insulin-Producing Cells Using Culture Conditions Favouring the Formation of Nestin-Postive Cells.

For differentiation of nestin-positive cells, mouse ES cells were cultivated for 2 days in hanging drops (100, 200, or 400 cells/drop) to form embryoid bodies (EBs; FIG. 10). EBs were then transferred to bacteriological petri dishes (Greiner, Germany) and cultivated for additional 2 days in Iscove's modification of DMEM (IMDM; Gibco) containing 20% FCS and supplements as described (Rohwedel et al., 1998), Dev. Biol. 201(2):167-184), with the exception that beta-mercaptoethanol was replaced by 450 mM alpha-monothioglycerol (Sigma, Steinheim, Germany). Between 20 and 30 EBs were plated onto tissue culture dishes (diameter 6 cm) at day 4, and cultivated in IMDM supplemented with 20% FCS for 24 hours. The selection of nestin-positive cells was carried out according to the method described by Okabe and colleagues (Okabe et al., 1996, Mech. Dev. 59:89-102) with the following modifications: After attachment of EBs (day 4+1), the medium was exchanged for a B1 medium prepared with a base of Dulbecco's modified Eagle's medium: Nutrient Mixture F-12 (DMEM/F12, Life Technologies) supplemented with 5 mg/ml insulin, 30 nM sodium selenite (both from Sigma), 50 mg/ml transferrin, and 5 mg/ml fibronectin (both from Gibco). The B1 culture medium was replenished every 48 hours. Nestin-positive cells were selected after cultivation for 7 days (=4+7d). At day 4+8, EBs were dissociated with 0.1% trypsin (Gibco)/0.08% EDTA (Sigma) in phosphate buffered saline (PBS) (1:1) for 1 min, collected by centrifugation, and replated onto poly-L-ornithine/laminin-coated tissue culture dishes containing a B2 medium prepared with a base DMEM/F12 supplemented with 10% FCS; 20 nM progesterone; 100 mM putrescine; 1 mg/ml laminin (all from Sigma); 25 mg/ml insulin; 50 mg/ml transferrin; 30 nM sodium selenite; B27 supplement; and 10 mM nicotinamide. This medium was replaced after 24 hours with B2 medium lacking FCS. At day 30 of plating; >75% of the Pax4 ES cells are positive for insulin compared to 20% for wild type ES cells (FIG. 11).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for differentiating stem cells into insulin-producing cells comprising:
culturing stem cells in a suitable medium and activating at least one gene involved in beta-cell differentiation.

2. The method of claim 1 further comprising:
aggregating said cultivated stem cells to form embryoid bodies, cultivating said embryoid bodies in a differentiation medium enhancing β-cell differentiation, identifying, and optionally selecting insulin-producing cells.

3. The method of claim 1, wherein said stem cells are selected from embryonic stem cells, adult stem cells, somatic stem cells and primordial germ cells.

4. The method of claim 1, wherein said stem cells are of human origin.

5. The method of claim 1, wherein the genes involved in β-cell differentiation are selected from the group consisting of Pdx1, Pax4, Pax6, ngn3, Nkx6.1, Nkx6.2, Nkx2.2, HB9, BETA2, NeuroD, IsI1, HNF1-alpha, HNF1-beta, HNF3, and combinations thereof.

6. The method of claim 5, wherein the genes are selected from Pdx1, Pax4, Pax6, ngn3, and combinations thereof.

7. The method of claim 5, wherein the genes are of human origin.

8. The method of claim 1, wherein the gene activation comprises a delivery of a pancreatic gene into stem cells.

9. The method of claim 8, wherein said gene delivery comprises a transfection of stem cells with a cDNA of at least one pancreatic gene under the control of a regulatory region allowing the initiation of transcription.

10. The method of claim 8, wherein said gene delivery comprises a DNA transfer using a viral delivery system.

11. The method of claim 1, wherein the gene activation comprises a delivery of a protein product of a pancreatic gene into stem cells.

12. The method of claim 2, wherein said embryoid bodies are formed by a hanging drop method.

13. The method of claim 2, wherein said differentiation medium is based on Iscove's modified Dulbecco's medium (IMDM) supplemented with fetal calf serum, L-glutamine, non-essential amino acids, and à-monothioglycerol optionally containing EGF, bFGF, progesterone, growth hormone, follistatin, and/or activin.

14. The method of claim 13, wherein said differentiation medium further contains extracellular matrix proteins, collagens, and/or mixtures of growth factors and extracellular matrix proteins.

15. The method of claim 1, wherein at least 20% insulin-producing cells are obtained after a differentiation time of 15 days.

16. The method of claim 15, wherein at least 40% insulin-producing cells are obtained.

17. The method of claim 1 further comprising a selection of nestin-positive cells.

18. The method of claim 1, wherein the insulin-producing cells are used for pharmaceutical applications.

19. The method of claim 18 for the treatment of pancreatic diseases, metabolic syndrome and metabolic disorders with impaired glucose levels, such as diabetes, hyperglycaemia, and/or impaired glucose tolerance.

20. The method of claim 18, wherein between 3000 and 100000 equivalent differentiated insulin-producing cells are administered per kilogram body weight.

21. A cell composition comprising insulin-producing cells obtainable by the method of claim 1.

22. The composition of claim 21 comprising at least 20% insulin-producing cells after a differentiation time of 15 days.

23. The composition of claim 22 comprising at least 40% insulin-producing cells.

24. The composition of claim 21 comprising a ratio of insulin-producing cells versus glucagon-producing cells of at least 2:1.

25. The composition of claim 24 comprising a ratio of at least 5:1.

26. The composition of claim 21 exhibiting an increase in the insulin secretion of at least 2-fold 15 min after stimulation with 27.7 mM glucose.

27. The composition of claim 21, which is a pharmaceutical composition.

28. The composition of claim 27 for the treatment of pancreatic diseases, metabolic syndrome and metabolic disorders with impaired glucose levels, such as diabetes, hyperglycaemia, and/or impaired glucose tolerance.

29. The composition of claim 27, which is administered by transplantation or used in a medical device.

30. A method for identifying and/or characterizing compounds capable of modulating the differentiation of stems cells into insulin-producing cells comprising: contacting a compound to be tested with stem cells under conditions wherein the stem cells are capable of being differentiated into insulin-producing cells and determining the effect of the compound on the differentiation process.

31. The method of claim 30 comprising transfecting stem cells with a DNA construct containing a reporter gene under regulatory control of a gene involved in β-cell differentiation, contacting said transfected cells with a compound to be tested and determining the activity of the reporter gene.

32. The method of claim 30 comprising contacting embryoid bodies which are cultivated in a differentiation medium enhancing β-cell differentiation with a compound to be tested and determining differentiation into insulin-producing cells.


SEQUENCE LISTING (20 sequences)

SEQ ID NO: 1 - DNA, 1122 bp, Mus musculus
  CDS (54)..(905)
cctggcttgtagctccgacccggggctgctggccccaagtgccggctgccaccatgaacagtgaggagcagtactacgcggccacacagctctacaaggacccgtgcgcattccagaggggcccggtgccagagttcagcgctaacccccctgcgtgcctgtacatgggccgccagcccccacctccgccgccaccccagtttacaagctcgctgggatcactggagcagggaagtcctccggacatctccccatacgaagtgcccccgctcgcctccgacgacccggctggcgctcacctccaccaccaccttccagctcagctcgggctcgcccatccacctcccggacctttcccgaatggaaccgagcctgggggcctggaagagcccaaccgcgtccagctccctttcccgtggatgaaatccaccaaagctcacgcgtggaaaggccagtgggcaggaggtgcttacacagcggaacccgaggaaaacaagaggacccgtactgcctacacccgggcgcagctgctggagctggagaaggaattcttatttaacaaatacatctcccggccccgccgggtggagctggcagtgatgttgaacttgaccgagagacacatcaaaatctggttccaaaaccgtcgcatgaagtggaaaaaagaggaagataagaaacgtagtagcgggaccccgagtgggggcggtgggggcgaagagccggagcaagattgtgcggtgacctcgggcgaggagctgctggcagtgccaccgctgccacctcccggaggtgccgtgcccccaggcgtcccagctgcagtccgggagggcctactgccttcgggccttagcgtgtcgccacagccctccagcatcgcgccactgcgaccgcaggaaccccggtgaggacagcagtctgagggtgagcgggtctgggacccagagtgtggacgtgggagcgggcagctggataagggaacttaacctaggcgtcgcacaagaagaaaattcttgagggcacgagagccagttggatagccggagagatgctgcgagcttctggaaaaacagccctgagcttctgaaaactttgaggctgcttctgatgccaagctaatgg

SEQ ID NO: 2 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 3 - DNA, 1084 bp, Mus musculus
  CDS (24)..(1019)
tgcgaggagtaccagtgtgaagcatgcagcaggacggactcagcagtgtgaatcagctagggggactctttgtgaatggccggccccttcctctggacaccaggcagcagattgtgcagctagcaataagagggatgcgaccctgtgacatttcacggagccttaaggtatctaatggctgtgtgagcaagatcctaggacgctactaccgcacaggtgtcttggaacccaagtgtattgggggaagcaaaccacgtctggccacacctgctgtggtggctcgaattgcccagctaaaggatgagtaccctgctctttttgcctgggagatccaacaccagctttgcactgaagggctttgtacccaggacaaggctcccagtgtgtcctctatcaatcgagtacttcgggcacttcaggaagaccagagcttgcactggactcaactcagatcaccagctgtgttggctccagttcttcccagtccccacagtaactgtggggctccccgaggcccccacccaggaaccagccacaggaatcgggctatcttctccccgggacaagccgaggcactggagaaagagtttcagcgtgggcagtatccagattcagtggcccgtgggaagctggctgctgccacctctctgcctgaagacacggtgagggtttggttttctaacagaagagccaaatggcgcaggcaagagaagctgaaatgggaagcacagctgccaggtgcttcccaggacctgacgataccaaaaaattctccagggatcatctctgcacagcagtcccccggcagtgtaccctcagctgccttgcctgtgctggaaccattgagtccttccttctgtcagctatgctgtgggacagcaccaggcagatgttccagtgacacctcatcccaggcctatctccaaccctactgggactgccaatccctccttcctgtggcttcctcctcatatgtggaatttgcctgccctgcctcaccacccatcctgtgcatcatctgattggaggcccaggacaagtgccatcatcccattgctcaaactggccataaccgcggaattcc

SEQ ID NO: 4 - protein, 332 aa, Mus musculus
MQQDGLSSVNQLGGLFVNGRPLPLDTRQQIVQLAIRGMRPCDISRSLKVSNGCVSKILGRYYRTGVLEPKCIGGSKPRLATPAVVARIAQLKDEYPALFAWEIQHQLCTEGLCTQDKAPSVSSINRVLRALQEDQSLHWTQLRSPAVLAPVLPSPHSNCGAPRGPHPGTSHRNRAIFSPGQAEALEKEFQRGQYPDSVARGKLAAATSLPEDTVRVWFSNRRAKWRRQEKLKWEAQLPGASQDLTIPKNSPGIISAQQSPGSVPSAALPVLEPLSPSFCQLCCGTAPGRCSSDTSSQAYLQPYWDCQSLLPVASSSYVEFACPASPPILCII

SEQ ID NO: 5 - DNA, 1872 bp, Mus musculus
  CDS (30)..(1337)
ggatccggaggctgccaaccagctccagcatgcagaacagtcacagcggagtgaatcagcttggtggtgtctttgtcaacgggcggccactgccggactccacccggcagaagatcgtagagctagctcacagcggggcccggccgtgcgacatttcccgaattctgcagacccatgcagatgcaaaagtccaggtgctggacaatgaaaacgtatccaacggttgtgtgagtaaaattctgggcaggtattacgagactggctccatcagacccagggcaatcggagggagtaagccaagagtggcgactccagaagttgtaagcaaaatagcccagtataaacgggagtgcccttccatctttgcttgggaaatccgagacagattattatccgagggggtctgtaccaacgataacatacccagtgtgtcatcaataaacagagttcttcgcaacctggctagcgaaaagcaacagatgggcgcagacggcatgtatgataaactaaggatgttgaacgggcagaccggaagctggggcacacgccctggttggtatcccgggacttcagtaccagggcaacccacgcaagatggctgccagcaacaggaaggagggggagagaacaccaactccatcagttctaacggagaagactcggatgaagctcagatgcgacttcagctgaagcggaagctgcaaagaaatagaacatcttttacccaagagcagattgaggctctggagaaagagtttgagaggacccattatccagatgtgtttgcccgggaaagactagcagccaaaatagatctacctgaagcaagaatacaggtatggttttctaatcgaagggccaaatggagaagagaagagaaactgaggaaccagagaagacaggccagcaacactcctagtcacattcctatcagcagcagcttcagtaccagtgtctaccagccaatcccacagcccaccacacctgtctcctccttcacatcaggttccatgttgggccgaacagacaccgccctcaccaacacgtacagtgctttgccacccatgcccagcttcaccatggcaaacaacctgcctatgcaacccccagtccccagtcagacctcctcatactcgtgcatgctgcccaccagcccgtcagtgaatgggcggagttatgatacctacacccctccgcacatgcaaacacacatgaacagtcagcccatgggcacctcggggaccacttcaacaggactcatttcacctggagtgtcagttcccgtccaagttcccgggagtgaacctgacatgtctcagtactggcctcgattacagtaaagagagaaggagagagcatgtgatcgagagaggaaattgtgttcactctgccaatgactatgtggacacagcagttgggtattcaggaaagaaagagaaatggcggttagaagcacttcactttgtaactgtcctgaactggagcccgggaatggactagaaccaaggaccttgcgtacagaaggcacggtatcagttggaacaaatcttcattttggtatccaaacttttattcattttggtgtattatttgtaaatgggcattggtatgttataatgaagaaaagaacaacacaggctgttggatcgcggatctgtgttgctcatgtggttgtttaaaggaaaccatgatcgacaagatttgccatggatttaagagttttatcaagatatatcaaatacttctccccatctgttcatagtttatggactgatgttccaagtttgtatcattcctttgcatataattgaacctgggacaacacacactagatatatgtaaaaactatctgttggttttccaaaggttgtt

SEQ ID NO: 6 - protein, 436 aa, Mus musculus
MQNSHSGVNQLGGVFVNGRPLPDSTRQKIVELAHSGARPCDISRILQTHADAKVQVLDNENVSNGCVSKILGRYYETGSIRPRAIGGSKPRVATPEVVSKIAQYKRECPSIFAWEIRDRLLSEGVCTNDNIPSVSSINRVLRNLASEKQQMGADGMYDKLRMLNGQTGSWGTRPGWYPGTSVPGQPTQDGCQQQEGGGENTNSISSNGEDSDEAQMRLQLKRKLQRNRTSFTQEQIEALEKEFERTHYPDVFARERLAAKIDLPEARIQVWFSNRRAKWRREEKLRNQRRQASNTPSHIPISSSFSTSVYQPIPQPTTPVSSFTSGSMLGRTDTALTNTYSALPPMPSFTMANNLPMQPPVPSQTSSYSCMLPTSPSVNGRSYDTYTPPHMQTHMNSQPMGTSGTTSTGLISPGVSVPVQVPGSEPDMSQYWPRLQ

SEQ ID NO: 7 - DNA, 861 bp, Mus musculus
  CDS (160)..(801)
attcttttgagtcgggagaactaggtaacaattcggaaactccaaagggtggatgaggggcgcgcggggtgtgtgtgggggatactctggtcccccgtgcagtgacctctaagtcagaggctggcacacacacaccttccattttttcccaaccgcaggatggcgcctcatcccttggatgcgctcaccatccaagtgtccccagagacacaacaaccttttcccggagcctcggaccacgaagtgctcagttccaattccaccccacctagccccactctcatacctagggactgctccgaagcagaagtgggtgactgccgagggacctcgaggaagctccgcgcccgacgcggagggcgcaacaggcccaagagcgagttggcactcagcaaacagcgaagaagccggcgcaagaaggccaatgatcgggagcgcaatcgcatgcacaacctcaactcggcgctggatgcgctgcgcggtgtcctgcccaccttcccggatgacgccaaacttacaaagatcgagaccctgcgcttcgcccacaactacatctgggcactgactcagacgctgcgcatagcggaccacagcttctatggcccggagccccctgtgccctgtggagagctggggagccccggaggtggctccaacggggactggggctctatctactccccagtctcccaagcgggtaacctgagccccacggcctcattggaggaattccctggcctgcaggtgcccagctccccatcctatctgctcccgggagcactggtgttctcagacttcttgtgaagagacctgtctggctctgggtggtgggtgctagtggaaagggaggggaccacagcc

SEQ ID NO: 8 - protein, 214 aa, Mus musculus
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 9 - DNA, 21 bp, Mus musculus
ttcggctatgacatcggtgtg

SEQ ID NO: 10 - DNA, 21 bp, Mus musculus
agctgaggccagcaatctgac

SEQ ID NO: 11 - DNA, 18 bp, Mus musculus
cagcccttagtgaccagc

SEQ ID NO: 12 - DNA, 18 bp, Mus musculus
atgctggtgcagcactga

SEQ ID NO: 13 - DNA, 18 bp, Mus musculus
cgcctgatcccttggatg

SEQ ID NO: 14 - DNA, 18 bp, Mus musculus
cagtcacccacttctgct

SEQ ID NO: 15 - DNA, 20 bp, Mus musculus
ctttcccgtggatgaaatcc

SEQ ID NO: 16 - DNA, 21 bp, Mus musculus
gtcaagttcaacatcactgcc

SEQ ID NO: 17 - DNA, 20 bp, Mus musculus
gtttgtacgggatcaaatgc

SEQ ID NO: 18 - DNA, 20 bp, Mus musculus
atgctgcgtttcttgtcctt

SEQ ID NO: 19 - DNA, 20 bp, Mus musculus
tcactgtgcctgaacttacc

SEQ ID NO: 20 - DNA, 20 bp, Mus musculus
ggaacatagccgtaaactgc